(19) United States
(12) Patent Application Publication
(10) Pub. No.: US 2015/0367083 A1
(43) Pub. Date: Dec. 24, 2015

(54) DISPENSING DEVICE FOR DISPENSING A FLUID

(71) Applicant: WEIBEL CDS AG, Waldstatt (CH)

(72) Inventor: Ludwig Daniel WEIBEL, Waldstatt (CH)

(21) Appl. No.: 14/765,860

(22) PCT Filed: Feb. 6, 2013
(86) PCT No.: PCT/EP2013/052296

§ 371 Date: Aug. 5, 2015

(51) Int. Cl. A61M 5/32; A61M 5/315

(52) U.S. Cl. CPC A61M 5/3204; A61M 5/31511

(57) ABSTRACT

A dispensing device (1) for dispensing a fluid comprising a base (2) having a longitudinal axis (A), a receiving chamber (14) formed in the base for the fluid, and a dispensing opening (18) formed in the base for dispensing the fluid from the receiving chamber. The dispensing opening (18) is formed in a dispensing area (4) of the dispensing device (1). Furthermore, the dispensing device (1) comprises a closure element which is movably arranged on the base. The closure element (30) comprises a dispensing channel (40) that is open to the outside. The closure element (30) is movable from a closed position, in which a fluid connection between the dispensing opening (18) and the dispensing channel (40) is interrupted, into a dispensing position, in which there is a fluid-tight fluid connection between the dispensing opening (18) and the dispensing channel (40). The dispensing device (1) includes a protective device (70).

The invention relates to a dispensing device for dispensing a fluid, in particular for medical use, preferably an injection syringe, according to the preambles of the independent claims.

Dispensing devices for dispensing a fluid include, for example, infusion devices, injection syringes and instillation syringes. Nowadays, for reasons of hygiene, disposable articles, such as disposable syringes, are mostly used. Disposable syringes are often delivered in a prefilled state. This has the disadvantage that the packaging size of such syringes is relatively large on account, of the piston rod being retracted in the filled state. Moreover, not ail medicaments in liquid form, are suitable for being stored in such disposable syringes over a long period of time. In particular, it is often necessary for different medical substances (e.g. liquid/liquid or liquid/solid) to be mixed or dissolved in each other shortly before being administered, since they are not able to be stored in mixture or in solution. For this purpose, the different medical substances have to be stored in separate containers and mixed under sterile conditions before they are transferred into a dispensing device, e.g. an injection syringe. There is therefore a need to create a dispensing device which ensures filling to be carried out from containers, e.g. glass vials, under conditions that are as sterile as possible. Such dispensing devices should also be suitable to be used just once and should provide a high degree of handling safety. Such devices can of course also be used in the non-medical sector, for example for chemical analysis purposes in the food sector or in the cosmetics sector.

PCT/EP2012/065955 describes a transfer system with a dispensing device for transferring a fluid from a container to the dispensing device. The dispensing device has a main, body with a receiving chamber for the fluid, which receiving chamber is firmly connected to and communicates with a cannula via a dispensing channel formed on the main body. By means of a valve device arranged rotatably on the main body, a filling channel can optionally be attached laterally to the dispensing channel. By way of a transfer adapter, a fluid can be delivered to the fluid channel, such that the dispensing device can be filled through the filling channel and via the dispensing channel, e.g. by being drawn up. By rotation of the valve device, the fluid connection of the filling channel to the dispensing channel can be interrupted after the filling procedure. It is in this way ensured that, when dispensing the fluid, the fluid passes through the dispensing channel into the cannula and does not leak through the filling channel. On account of the fixed fluid connection between cannula and receiving chamber via the dispensing channel, which connection exists independently of a fluid connection of the filling channel, external air often passes through the dispensing channel into the receiving chamber of this dispensing device during the filling procedure, in particular during the drawing up. An at any rate conceivable filling procedure by forcing the fluid into the filling channel is likewise ruled out, since in this case the fluid would escape through the dispensing channel and the cannula.

It is therefore the object of the invention to overcome the disadvantages of the prior art. In particular, it is an object of the invention to make available a dispensing device, in particular an injection syringe, which is simple and safe to handle and is preferably intended for single use. The dispensing device should also foe versatile in use, particularly also in connection with a transfer system for transferring a fluid.

These objects are achieved by the features of the independent claims. In a first, aspect of the invention, these concern a dispensing device for dispensing a fluid, in particular for medical use, preferably an injection syringe. The fluid preferably comprises a medicament. The dispensing device comprises a main body with a longitudinal axis, and a receiving chamber formed in the main body for the fluid. The dispensing device comprises a dispensing opening, formed on the main body, for dispensing the fluid from, the receiving chamber, wherein the dispensing opening is formed in a dispensing area of the dispensing device. The dispensing device further comprises a closure element, which is arranged on the main body in such a way as to be rotatable relative to the latter about a rotation axis in the dispensing area, in particular a rotation axis parallel to the longitudinal axis of the main body. The dispensing device is characterized in that the closure element comprises a dispensing channel that is open to the outside. The closure element can be brought by rotation from a closed position, in which a fluid connection between the dispensing opening and the dispensing channel is interrupted, to a dispensing position, in which there is an in particular fluid-tight and preferably direct fluid connection between the dispensing opening and the dispensing channel.

The main body of the dispensing device can be of tubular shape, wherein the receiving chamber, can be designed as a circular cylindrical cavity. Typically, the main body has a closure piece at one end, at which the receiving chamber is closed off in the dispensing area. The dispensing opening is generally arranged on the closure piece. A piston is generally arranged in the receiving chamber and movable in the longitudinal direction. The piston is in most cases connected rigidly to a piston rod which protrudes outward at an open longitudinal end of the main body opposite the dispensing area and, for example, has a finger plate allowing the piston to be moved by a user.

The piston can have a sealing element, which seals off an area between the piston and a side wall of the receiving chamber. An overpressure can be generated in the receiving chamber with the piston, such that, for example, a fluid present in the receiving chamber can be dispensed through the dispensing opening (movement of the piston toward the dispensing opening). Likewise, an underpressure can be generated in the receiving chamber with the piston, such that a fluid can be introduced through the dispensing opening into the receiving chamber (movement of the piston away from the dispensing opening).

The closure element is preferably mounted movably on the main body. The closure element can be mounted so as to be slidable or rotatable on the main body. For this purpose, for example, guide means, e.g. grooves or rotary bearings, can be present, which ensure a secure fit of the closure element on the main body.

The closure element comprises a dispensing channel which is open to the outside, i.e. has a mouth which communicates with the environment when the closure element is arranged ready for operation on the main body. By a movement, of the closure element relative to the main body, the dispensing channel can be brought to the closed position in which a fluid connection between the dispensing opening of the main body and the dispensing channel is interrupted. The dispensing opening can thus be closed with the closure element, such that, for example, a fluid present in the receiving chamber cannot be dispensed. Likewise, the closure element can be brought to the dispensing position, in which the fluid connection between the dispensing opening of the main body and the dispensing channel is produced, in the dispensing positron, the fluid can be dispensed outward from the receiving chamber through the dispensing opening and the dispensing channel. The fluid connection is preferably fluid-tight, such that all of the fluid passes from the dispensing opening into the dispensing channel. The fluid connection can preferably be produced by direct attachment of the dispensing channel to the dispensing opening, i.e. the dispensing channel connects directly to the dispensing opening of the main body without any further fluid-guiding elements being arranged between them. It will be appreciated that sealing elements may be present that ensure the fluid tightness.

In this way, a dispensing device is made available in which the dispensing of the fluid from the receiving chamber can be alternately blocked or unblocked.

The closure element is preferably arranged on the main body in such a way as to be rotatable about a rotation axis in the dispensing area, in particular a rotation axis parallel to the longitudinal axis of the main body, wherein the closure element is movable by rotation between the closed position and the dispensing position. For this purpose, the closure element can be mounted on a bearing stub, formed on the main body, so as to be rotatable about a rotation axis parallel to the longitudinal axis of the main body. The bearing stump is preferably arranged on the closure piece of the main body in the dispensing area and extends away from the main body in the longitudinal direction. The dispensing opening is advantageously arranged on a front face of the bearing stub. In variants, the closure element can enclose the outside of the main body e.g. in the manner of a cuff, which is also conceivable in the case of a slidable closure element. The dispensing channel on the closure element preferably opens in the direction of the longitudinal axis toward the mart body at an inlet opening and away from the main body at an outlet opening. The outlet opening communicates at the same time with the environment.

Advantageously, the dispensing channel in the closure element, likewise extends, at least in the respective mouth area, parallel to the longitudinal axis. In this way, the fluid delivered or dispensed through the respective mouth can enter or leave the dispensing channel in a manner substantially free of turbulence. The dispensing channel can extend in the closure element rectilinearly and wholly parallel to the rotation axis of the closure element, in particular to the longitudinal axis of the main body.

The outlet opening, in particular also the dispensing channel if appropriate, is preferably arranged concentrically with respect to the longitudinal axis, in particular the rotation axis. If is thus easily ensured that, with a similarly eccentric arrangement of the dispensing opening on the main body, the fluid connection between dispensing opening and dispensing channel can be produced or interrupted by rotation of the closure element.

Advantageously, a cannula is arranged or can be arranged on the closure element in such a way that there is a fluid-tight fluid connection to the dispensing channel, wherein preferably the cannula is firmly fitted in the dispensing channel and is in particular arranged parallel to the longitudinal axis or the closure element.

Cannula designates a hollow needle with an interior designed to guide a fluid. The cannula in the present case includes in particular an injection needle for the subcutaneous, intravascular or intramuscular introduction of a medicament into the human or animal body (by injection) or for the removal of liquids from, the body (by puncturing). The term, also includes what are called irrigation cannulas, in which the fluid can be injected for example into existing openings (body cavities) without damaging the tissue.

The cannula can be plugged or screwed, e.g. by means of a Luer connection, onto a corresponding Luer cone on the closure element. However, the cannula is preferably inserted directly into the dispensing channel and, for example, adhesively bonded. The closure element is preferably formed as an injection molded part. In this case, the cannula can be directly encapsulated during the injection of the closure element. For a secure attachment, the cannula can extend all the way along the dispensing channel as far as the inlet opening.

The closure element preferably has a portion with a substantially frustoconical shape, with a jacket surface narrowing away from the main body in a cone shape with respect to the longitudinal axis. A top face of the frustoconical portion preferably forms a front face of the closure element. The outlet opening of the dispensing channel can open out on the front face. The inlet opening can be arranged on a bottom face, wherein a receiving chamber for the bearing stub of the main body is at any rate present on the bottom face. In this way, a structure narrowing toward an attached cannula is created in the dispensing area.

Advantageously, the closure element comprises a filling channel and can be arranged in a filling position in which there is an in particular fluid-tight fluid connection between the dispensing opening and the filling channel. The filling position is preferably a closed position in which a fluid connection between the dispensing opening and the dispensing channel is interrupted. A fluid, e.g. from an external filling device, can be introduced into the receiving chamber via the filling channel. The dispensing opening of the main body serves here as a filling opening through which the fluid from the filling device can pass through the filling channel into the receiving chamber. The fluid can be forced from the outside into the receiving chamber or can be drawn up in the receiving chamber by generating an underpressure. The closure element thus acts as a valve device for alternately connecting the dispensing opening to the filling channel or the dispensing channel.

The filling channel preferably opens outward at a filling opening on a radial outer surface with respect to the longitudinal axis, in particular, and if appropriate, on the jacket surface narrowing in a cone shape. In this way, the fluid can be delivered laterally to the filling channel with respect to the longitudinal axis, i.e. if appropriate also with respect to the rotation axis of the closure element. This has the advantage that, even with a cannula inserted for example into the front end of the dispensing channel, the dispensing device can be filled unimpeded with a fluid. The filling channel advantageously extends, at least in a mouth area, in a radial, direction with respect to the longitudinal axis and, if appropriate, the rotation axis.

A blocking device is advantageously preset with which a relative movement direction, in particular a rotation direction, of the closure element relative to the main body is predefined. The predefined movement direction in the case of a filling channel is preferably oriented from the filling position to the dispensing position. It is ensured in this way that a user cannot move, e.g. rotate, the closure element in the wrong direction after a filling procedure. The blocking device can be designed here in the manner of a ratchet and can, for example, comprise pawls on the closure element which engage in corresponding teeth on the main body, or vice versa.

Preferably, a movement range, in particular a rotation range, of the closure element is in addition limited by a limiting device in such a way that a movement is possible only as far as the dispensing position. In this way, it is ensured that a user cannot move the closure element too far. Moreover, in combination with the blocking device, it is ensured that the closure element is locked in the dispensing position once the latter has been reached. The closure element can thus no longer be brought to the filling position, as a result of which repeated use of the dispensing device can be prevented.

The limiting device can, for example, comprise abutments and mating abutments on closure element and main body, which abutments abut each other when the dispensing position, is reached. Likewise, a guide can be provided on the main body for example, in which guide a pin of the closure element is movable in a guided, manner, e.g. within a limited rotation range.

The closure element preferably comprises a coupling means, wherein in particular the frustoconical portion is designed as a coupling means, for coupling the dispensing device to a further device. The further device can be, for example, a filling device or a transfer device for transferring a fluid, or also simply a protective cap for receiving the cannula. The further device preferably has a seat for the coupling means.

The coupling means preferably comprises, for example, securing means for securing the coupling. These can be, for example, ribs and/or notches that secure a plugged connection. Likewise, a means of securing against rotation can be present, e.g. in the form of longitudinal grooves, by which a rotation of the coupling means, or of the closure element rigidly connected thereto, is prevented in the seat. It will be appreciated that the seat of the further device may have complementary securing means.

Advantageously, a locking means is present on the dispensing device, particularly in the manner of a bayonet, with which the dispensing device can be locked on a complementary locking means of a further device. The locking means is preferably arranged on the main body. Particularly in the case where the closure element comprises a coupling means, the locking means is preferably designed in suck a way that the locking means permits locking in the direction of introduction and/or in the direction of removal from the seat of the further device. Particularly in the case of a rotatable closure element, the locking means preferably comprises a part of a bayonet-like closure. A complementary part of the bayonet-like closure is in this case formed on the further device. The rotation needed to release the locking means is preferably such that, upon corresponding rotation of the closure element in the opposite direction, a transfer from the filling position to the dispensing position takes place. In this way, with a coupling means arranged securely against rotation in the seat, the rotation of the main body for releasing the locking means causes the counter-rotation of the closure element relative to the main body, as is necessary for the transfer to the dispensing position.

The dispensing device preferably comprises a protective cap, in particular with a receiving chamber for receiving the cannula, which protective cap has a seat for the coupling means of the closure element. In this way, the protective cap can be fitted easily onto the coupling means.

Advantageously, the protective cap comprises an attachment nozzle for attaching a source of fluid, and a transfer channel for transferring a fluid from the attachment nozzle to an orifice on the seat. In this way, the protective cap can at the same time perform a function as a transfer device which, during filling of the dispensing device, can be attached for example to a source of fluid or a vial. For this purpose, the orifice of the transfer channel is preferably designed and arranged on the seat in such a way that, when coupling means of the closure element are arranged in the seat, there is an in particular fluid-tight fluid connection between the orifice on the seat and the filling opening of the filling channel. The protective cap or the attachment nozzle can be designed in such a way that it is attachable to known systems for transfer of a fluid.

The seat of the protective cap and the coupling means of the closure element can have complementary positioning means which allow the coupling means to be introduced into the seat only in one orientation. Introduction is preferably possible in an orientation in which the orifice of the transfer channel and the filling opening of the filling channel join each other in a fluid-tight manner.

The protective cap preferably has a looking means complementary to the locking means of the dispensing device, which locking means of the protective cap is designed, in particular with ramp-shaped elements, in such a way that, when, the locking action is released, in particular when the main body is rotated with respect to the protective cap, the coupling means is forced at least partially from the seat. It has been found that the coupling means can be arranged in the seat of the protective cap in such a way that withdrawal can be difficult even after the locking has been released. In particular, sealing ribs present on the coupling means, which engage in sealing grooves of the seat, can for example counteract simple removal of the dispensing device. Since ramp-shaped elements, for example, are present on the locking means of the protective cap, and since the locking means of the dispensing device slide onto the ramp-shaped elements by rotation when the locking action is released, the coupling means can be lifted at least slightly from the seat. Thereafter, removal of the protective cap is readily possible.

In order to further improve the safety of use of the dispensing device with a cannula, the dispensing device preferably comprises a protective device with a rigid protective body for the needle. However, a protective device of this kind is also generally advantageous in dispensing devices that have no closure element.

A further aspect of the invention therefore concerns a dispensing device for dispensing a fluid, in particular for medical use, preferably an injection syringe, in particular a dispensing device as described above, comprising a main body with a longitudinal axis, and a receiving chamber formed in the main body for the fluid. The dispensing device further comprises a dispensing area, which is designed for the arrangement of a cannula communicating with the receiving chamber, or in which a cannula communicating with the receiving chamber is arranged, and through cannula which the fluid can be dispensed from the receiving chamber. The cannula is in particular an injection needle.

The dispensing device comprises a protective device with a rigid protective body arranged on the main body. The protective device is preferably designed to cover a cannula after a fluid has been dispensed from, the dispensing device.

The protective body is designed in such a way and is movable with respect to the main body between a standby position and a protective position in such a way that the cannula arranged or able to be arranged in the dispensing area is completely exposed in the standby position, and the protective body, in the protective position, extends from the main body completely over the cannula arranged or able to be arranged in the dispensing area.

Typically, a needle that can be arranged in the dispensing area is completely exposed in the standby position when a coupling site for coupling the cannula is at least partially freed by the protective body. Since such cannulas often have standard lengths, it is immediately apparent how far the protective body has to extend beyond the main body in the protective position in order to protrude fully beyond the cannula, even when the latter is not arranged fixedly on the dispensing device.

Depending on the intended application, cannulas with lengths in the range of approximately 10 mm to 120 mm can typically foe used. For example, in the case of insulin injections, comparatively snort cannulas with lengths of approximately 10 mm to 20 mm are typical, whereas in the dental sector, for example, lengths of approximately 10 mm to 45 mm are used. In the case of intramuscular, subcutaneous, intravenous or intra-arterial injections, the lengths of the cannulas are typically in the range of 20 mm to 80 mm. In neural therapy, it is even possible for cannulas with lengths of up to 120 mm to be used. Depending on the cannulas with which the dispensing device is intended, to be used, the protective body extends so far beyond the main body in the protective position, that it is able to protrude completely past the cannulas that are envisioned for use.

A trigger means is provided which can be triggered directly or indirectly by a user and with which a transfer of the protective body from the standby position to the protective positron can be triggered. A securing means is present with which the protective device can be brought to a secured state and an unsecured state, wherein the protective device in the secured state is secured against a triggering of the transfer from the standby position to the protective position, and, in the unsecured state, the transfer can be triggered.

The protective body can nave a U-shaped, tubular or sleeve-shaped configuration, such that the cannula, in the protective position, is arranged in an interior of the protective body at least partially enclosed by the protective body. The cannula is protected in this way, such that a user can no longer suffer an injury from the cannula. The protective body can be designed in such a way that, in the standby position, the main body is at least partially enclosed by the protective body. The protective body is therefore preferably designed as a sleeve which, upon transfer to the protective position, can be moved in the longitudinal direction of the main body over the dispensing area and the cannula. In relation to the main body, the protective body is preferably subject to a spring force in such a way that, when the trigger means is triggered, the transfer to the standby position takes place automatically. A spring is provided for this purpose, preferably in the dispensing area, which spring acts between the main body and the protective device and subjects the protective body to a spring force in the direction of the protective position.

The protective device preferably comprises locking means which lock the protective body on the main body in the protective position once the latter has been adopted, such that the dispensing device can no longer be used. For this purpose, the protective body can comprise pawls, for example, which interact with a latching edge of the main body in the protective position, in such a way that the protective body can no longer be brought to the standby position.

The trigger means can, for example, comprise a releasable locking means which can be released when the dispensing device is used to dispense the fluid. The trigger means is to be distinguished from the securing means, which firmly locks the protective body in the secured state and secures it against transfer to the protective position. According to this aspect of the invention, the trigger means can be triggered, only in the unsecured state.

The securing means is advantageous in particular in dispensing devices which are filled, i.e. charged for example, with a fluid just shortly before use. With the additional scouring means, it is ensured that the protective device cannot be accidentally triggered before the dispensing device is in fact in a state ready for dispensing. The securing means is preferably designed in such a way that the protective device is automatically in the unsecured state during or after the filling procedure, i.e. in particular as soon as it is in a state ready for dispensing.

The securing means is preferably designed in such a way that, in the secured state of the protective device, the trigger means is secured by the securing means against being triggered. Since the securing means secures the trigger means against being triggered, it is easily ensured that no triggering can take place. The securing means preferably acts on the trigger element counter to a trigger direction. In variants, the securing means can also act on the protective body and lock the latter, such that the trigger means can be triggered bat the protective body cannot be moved to the protective position.

Advantageously, the trigger means comprises at least one latching tongue which is formed on the protective body and, in the standby position of the protective body, is latched on a latching edge of the main body. The latching edge can be formed on a longitudinal end of the main body opposite the dispensing area. The latching tongue preferably engages wish a latching projection around the latching edge in such a way that, e.g. from the outside, triggering can take place simply by bending the latching tongue away from the main body.

The dispensing device can have an actuating device with a piston which is movable in the receiving chamber and which is movable from an inserted position to a retracted position in order to introduce the fluid into the receiving chamber and is movable in the opposite direction in order to discharge the fluid. The receiving chamber and the piston typically match each other with a circular cylindrical shape, wherein the piston may at most be provided with a radially acting seal in order to seal off a tolerance gap that is present between the piston and the inside wall of the receiving chamber and that allows mobility.

The actuating device can comprise a piston rod which protrudes outward from the receiving chamber at an open longitudinal end of the main body opposite the dispensing area and which, for example, has a finger plate allowing a user to move the piston. By pulling the finger plate in the longitudinal direction, the dispensing device can be charged, i.e. the piston can be brought to a retracted position. By pressing the finger plate in the longitudinal direction, the fluid can be forced out through the dispensing opening, wherein the piston can be brought to the inserted position.

In this context, “retracted position” designates various positions in which the piston is moved out of the inserted position away from the dispensing opening. By contrast, inserted position designates a position in which the piston is moved substantially completely in the receiving chamber toward the dispensing opening.

The actuating device, or a part of the actuating device, is preferably formed as a securing means in such a way that the protective device can be brought to the secured state in the inserted position of the piston. In this way, the protective device can be in a secured state when the dispensing device, particularly prior to use, contains no fluid or has not yet been charged.

The actuating device is preferably formed in such a way that, upon transfer of the piston from the retracted position to the inserted position, the trigger means can be triggered, in particular coercively. It is in this way ensured that, when dispensing the fluid, i.e. when performing an injection for example, a transfer of the protective device to the protective position can be triggered automatically.

Advantageously, the securing means is formed in such a way that, in a retracted position of the piston, the protective device is by necessity in the unsecured state. That is to say, as soon as the dispensing device has been fully or partially charged, the protective device is in an unsecured state. It is ensured in this way that, as soon as the dispensing device is in a state ready for dispensing, the protective device is in an unsecured state and can be triggered. The securing means is preferably designed in such a way that, after a transfer from the secured state to the unsecured state of the protective device, the secured state can no longer be produced.

The actuating device preferably has a finger plate for moving the piston, which finger plate is in particular connected rigidly to the piston and is designed as a securing means. Since the finger plate is designed as a securing means, the dispensing device can be designed particularly simply and without, additional parts. When moving the piston to a retracted position, the finger plate is moved away from the trigger means, whereby an operative connection of the securing means to the trigger means is interrupted, such that the latter is transferred to an unsecured state. The transfer is preferably irreversible, such that the secured state can no longer be produced (see below).

The securing means is preferably designed as at least one passage which is formed in the finger plate and through which the trigger means, in particular the at least one latching tongue if appropriate, can extend when the piston is in the inserted position. The passage is preferably designed in such a way that the latching tongue is blocked against triggering as long as it protrudes through the passage.

For this purpose, the passage advantageously has an abutment that, blocks the trigger means in a trigger direction. In the case of a latching tongue, the abutment is preferably arranged with respect to the latching tongue, and also to the latching edge of the main body, in such a way that the latching tongue can be fixed on the latching edge by the abutment. In this way, the abutment of the passage forms, with respect to the latching edge, a counterbearing for the latching tongue, which counterbearing prevents triggering. In other words, the abutment of the passage fixes the trigger means, in particular the latching tongue, in the trigger direction thereof, in particular with respect to the latching edge, in such a way that triggering is not possible.

The securing means and/or the trigger means preferably has a blocking means, which is designed in such a way that restoration of the operative connection, in particular between securing means and trigger means, of the secured state is prevented once this operative connection has been canceled.

Particularly in the case of a latching tongue, the blocking means can preferably be designed in such a way that the latching tongue, although removable in one direction through the passage of the finger plate, is blocked in the opposite direction against introduction into the passage. For this purpose, the latching tongue, preferably at an area protruding through the passage in the secured state, has a blocking means which is designed, as a preferably deformable widening. The widening preferably extends, in a projection along the movement direction of the finger plate, beyond a clear cross section of the passage.

The widening can be released from the passage, e.g. by the widening being deformed. Likewise, the widening can be deflected in such a way that it can be brought into an area of the passage in which it does not overlap with the passage or can be guided through it.

After the latching tongue is released from the passage, the widening can it appropriate return again to the originally overlapping shape, e.g. on account of elastic deformation. When it has been deflected, the latching tongue can for example be restored to the original position, i.e. to an area of tire passage in which the widening overlaps with this (e.g. automatically on account of a resilient action of the latching tongue). As a result of the widening, the latching tongue can thus no longer enter the passage if the finger plate or the passage is moved in the opposite direction, i.e. preferably toward the main body.

The widening can be formed running out, for example, toward the free end of the latching tongue, in order to ensure a simple release from the passage in one direction and to ensure a blocking against introduction in the other direction. For this purpose, the widening can be designed spreading in the blocking direction. A deformation gap can be present on the widening, for example, such that portions separated by the deformation gap can be pressed together during release from the passage. In the opposite direction, by contrast, the two portions can be spread open for blocking on account of the deformation gap.

In variants, it is in principle also conceivable to provide the trigger means with a tamper-evident tab having a predetermined break point, which can be torn off at the transfer from the secured state to the unsecured state. In this case, the missing tamper-evident tab preferably prevents the securing means from interacting with the trigger means to restore the secured state. This can be achieved in a simple way, for example, if the securing means interacts with the trigger means only via the tamper-evident tab. It will be appreciated that this embodiment is not restricted to at design of the trigger means as latching tongue and instead can also be generally advantageous in other embodiments of the trigger means.

The actuating device preferably has a control face which is formed preferably on the finger plate, in particular on a side directed toward the main body, and which, during the transfer of the piston from a retracted position to the inserted position, cooperates with the trigger means, in particular the latching tongue, in such a way that the trigger means is movable or is moved from the control face in a triggering direction, wherein in particular a latching of the latching tongue on the latching edge of the main body can be released.

When the latching tongue, in the standby position of the protective device, engages around an edge of the main body for example, the control face can be formed, for example, extending outward from a piston rod toward the finger plate. In this way, the latching tongue is forced outward when the finger plate is moved toward the main body. It will be immediately apparent how the trigger means can be triggered in another embodiment and arrangement, since a movement of the finger plate or of other parts of the actuating device is converted into a movement of the trigger means in the triggering direction.

It will be appreciated that several trigger means, in particular several latching tongues, may also be present. Accordingly, several securing means, in particular passages, may also be present on the finger plate and, in the secured state, secure each trigger means against being triggered. If appropriate, in the case of several trigger means, it is however also sufficient to secure just, one of the trigger means against being triggered.

The invention is explained in detail below on the basis of illustrative embodiments depicted schematically in the figures, in which:

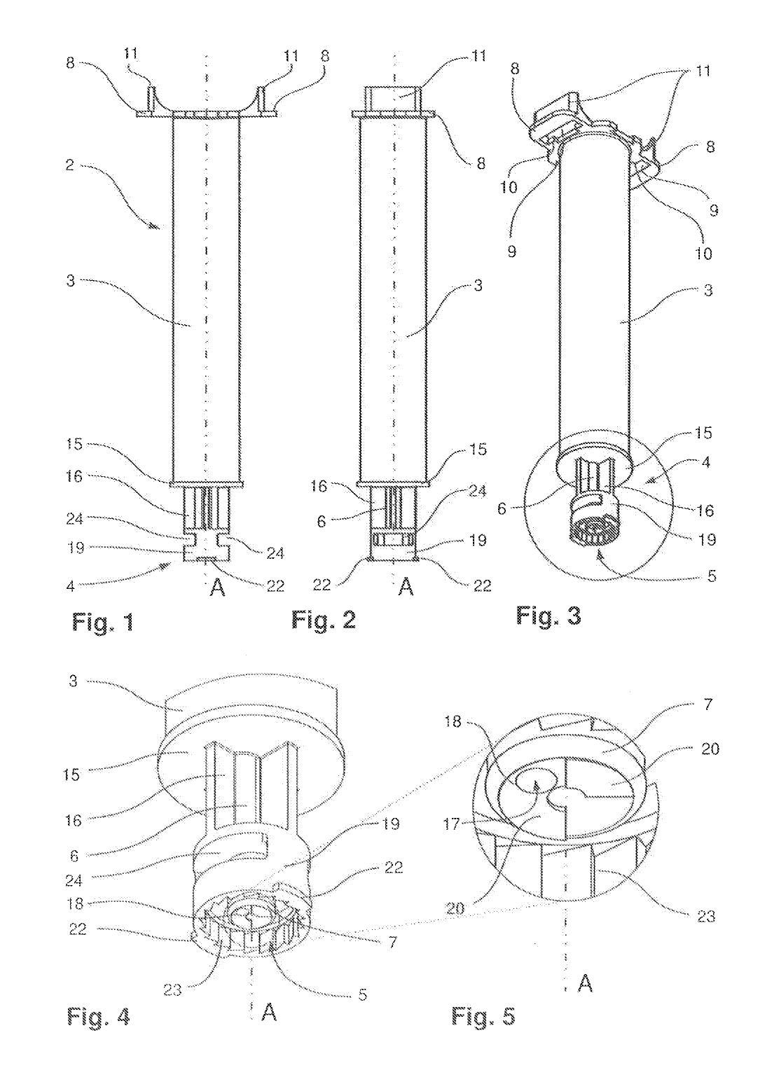

FIG. 1 shows a side view of a main body of a dispensing device according to the invention;

FIG. 2 shows a further side view of the main body according to FIG. 1;

FIG. 3 snows an oblique view of the main body according to FIG. 1;

FIG. 4 shows a detail of a dispensing area of the main body from FIG. 1;

FIG. 5 shows a further detail of the dispensing area of the main body from FIG. 1;

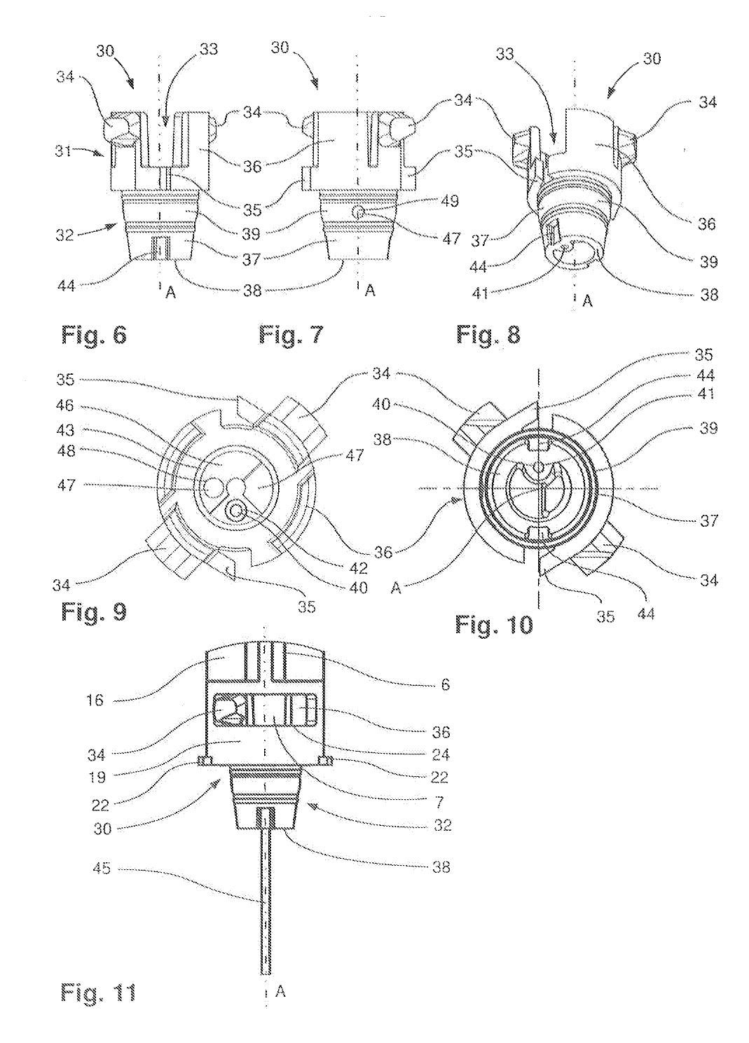

FIG. 6 shows a side view of a closure element for a dispensing device according to the invention;

FIG. 7 snows a further side view of the closure element according to FIG. 6;

FIG. 8 shows an oblique view of the closure element according to FIG. 6;

FIG. 9 stows a plan view of the closure element from FIG. 6 along a longitudinal axis;

FIG. 10 shows a further plan view of the closure element from FIG. 6 along a longitudinal axis, counter to the viewing direction of FIG. 9;

FIG. 11 shows a side view of the dispensing device with the closure element arranged in the dispensing area;

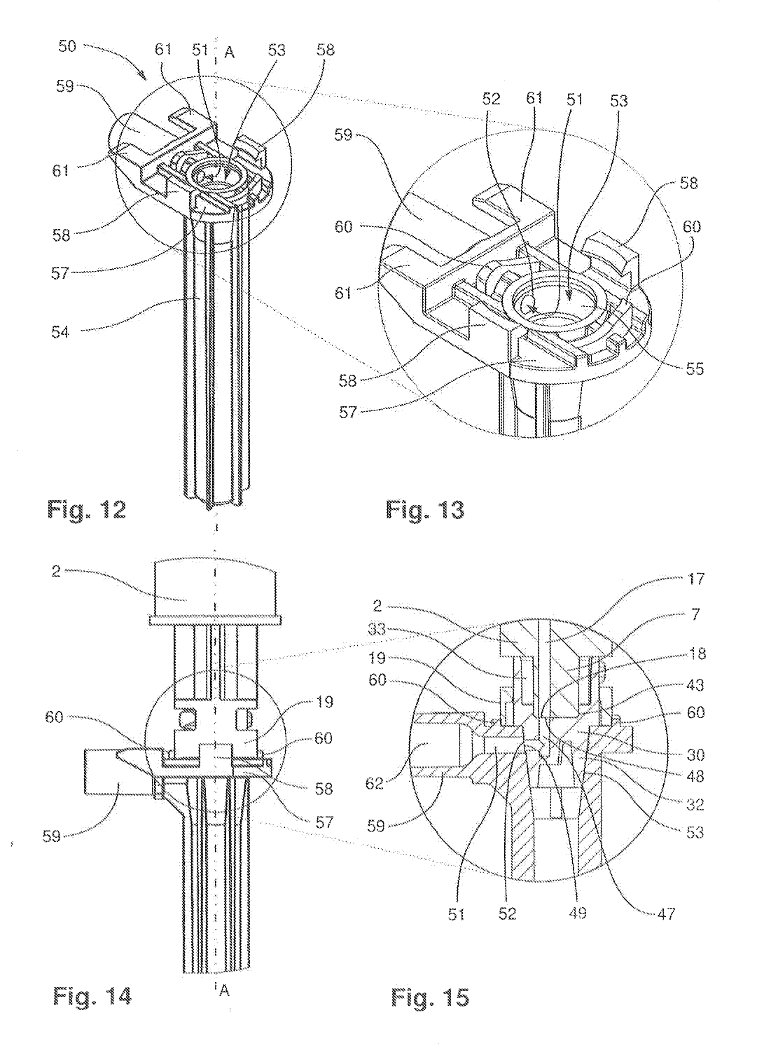

FIG. 12 shows a protective cap for a dispensing device with a transfer channel for delivery of a fluid;

FIG. 13 shows a detail of a seat of the protective cap from FIG. 12 for a coupling means of the dispensing device;

FIG. 14 shows a detail of the dispensing area of the dispensing device provided with the protective cap, with the closure element in a filling position;

FIG. 15 shows a partial view of a cross section of the view from FIG. 14;

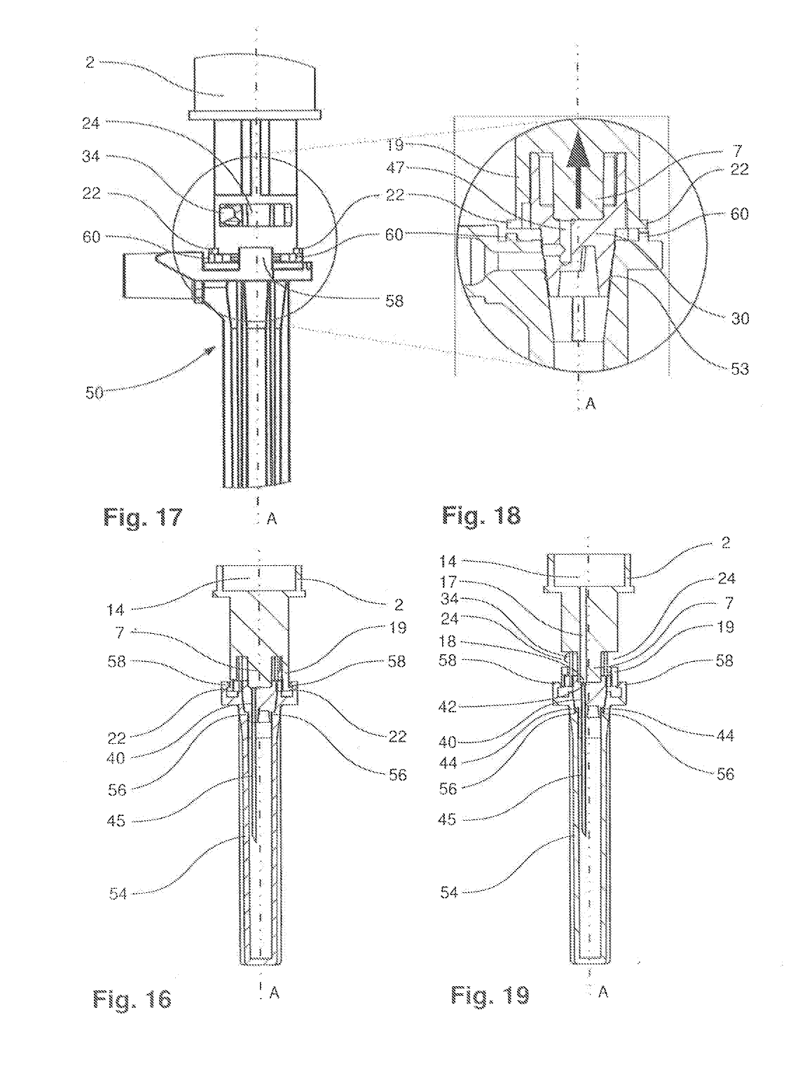

FIG. 16 shows a cross-sectional view of the view from FIG. 14, in a plane perpendicular to the view from FIG. 15;

FIG. 17 shows the detail according to FIG. 14, wherein the closure element is in a dispensing position, shortly before the removal from the protective cap;

FIG. 18 shows a partial view of a cross section of the view from FIG. 17;

FIG. 19 shows a cross-sectional view of the view from FIG. 17, in a plane perpendicular to the view from FIG. 18;

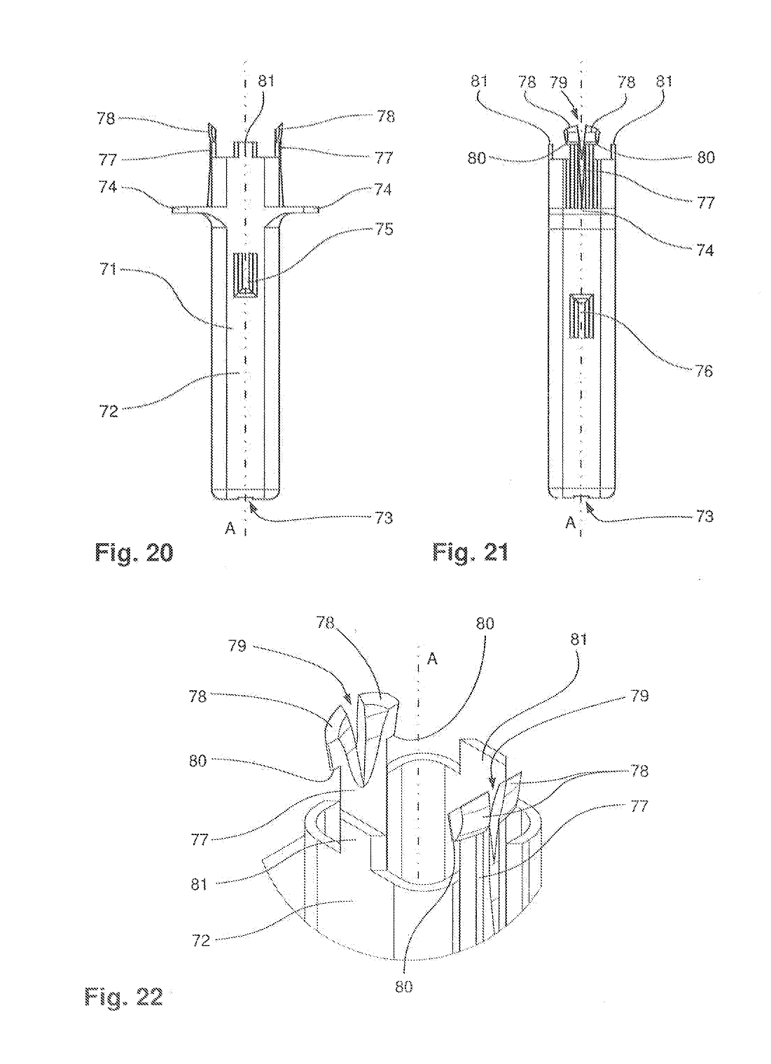

FIG. 20 shows a side view of a protective body of a protective device;

FIG. 21 snows a further side view of the protective body from FIG. 20;

FIG. 22 shows a detail of trigger means of the protective body from FIG. 20;

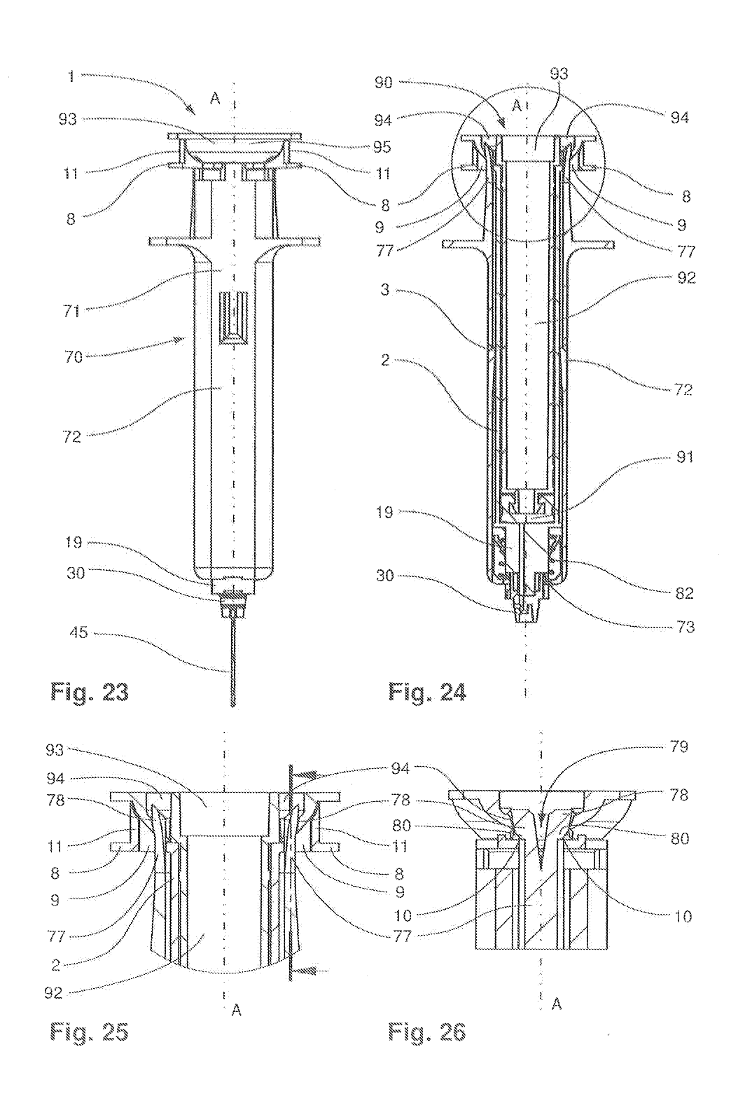

FIG. 23 shows an external view of a dispensing device with a main body and with a protective device in a secured state;

FIG. 24 shows a cross-sectional view of the view from FIG. 23;

FIG. 25 shows a detail of the view from FIG. 24 in the area of the trigger means of the protective device;

FIG. 26 shows a cross-sectional view in a section plane according to FIG. 25;

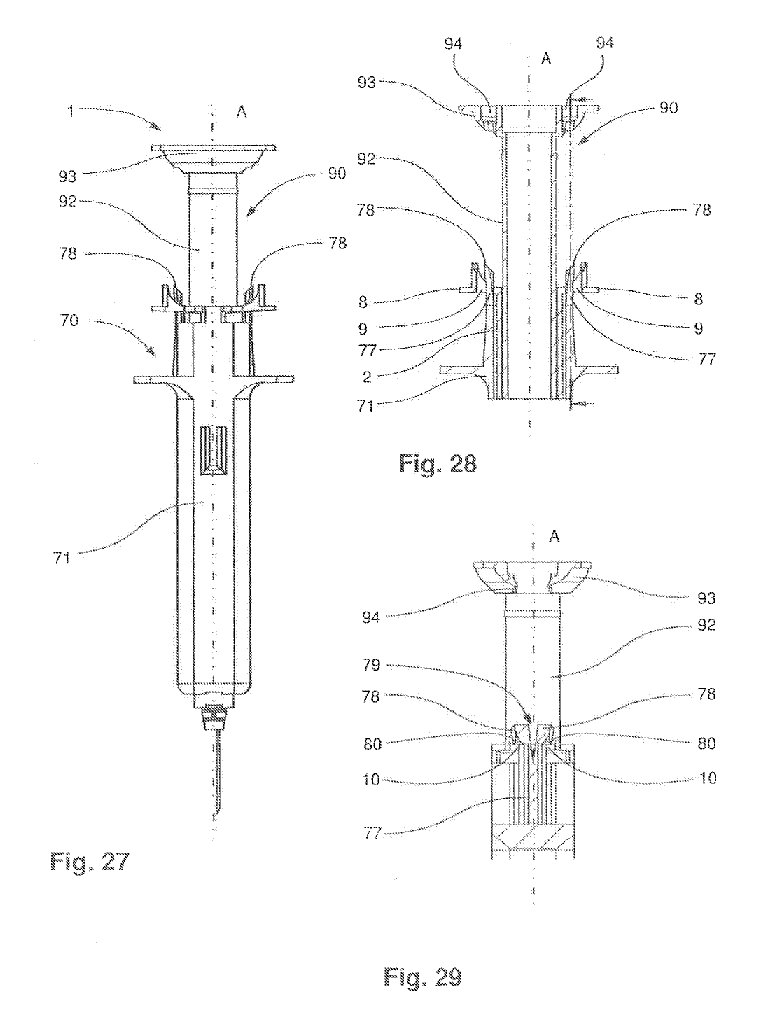

FIG. 27 shows an external view of the dispensing device from FIG. 23, in an unsecured state;

FIG. 28 shows a partial view of a cross section of the view from FIG. 27;

FIG. 29 shows a cross-sectional view in a seer ion plane according to FIG. 28;

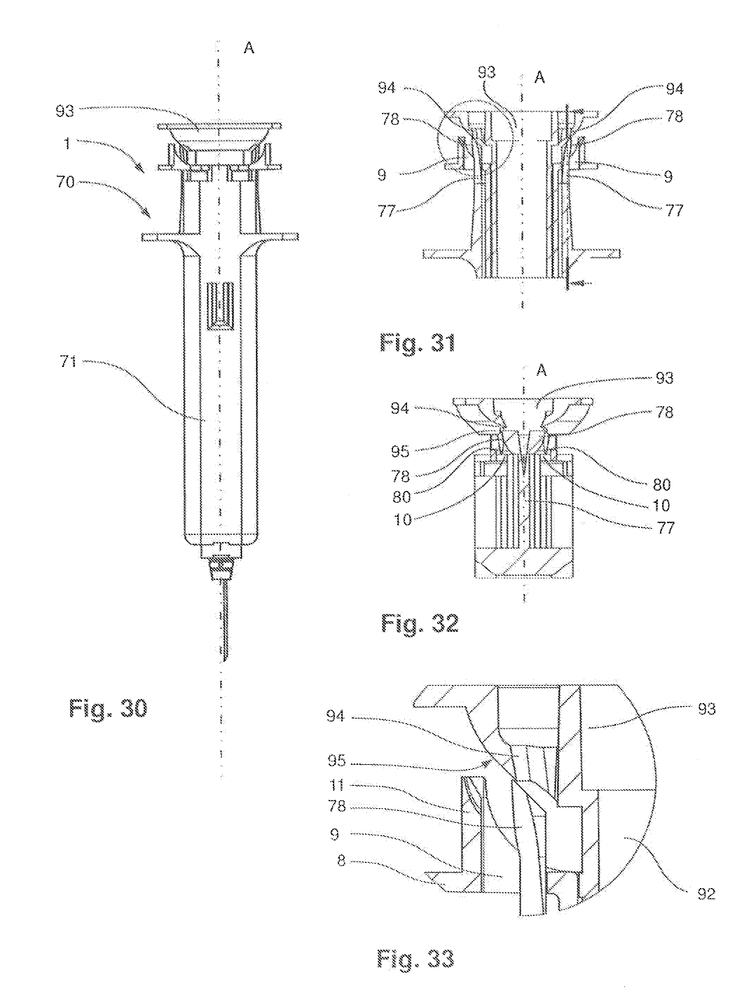

FIG. 30 shows an external view of the dispensing device from FIG. 23 at the moment the protective device is triggered;

FIG. 31 shows a partial view of a cross section of the view from FIG. 30 in the area of the trigger means;

FIG. 32 shows a cross-sectional view in a section plane according to FIG. 32;

FIG. 33 shows a detail of the view from FIG. 32;

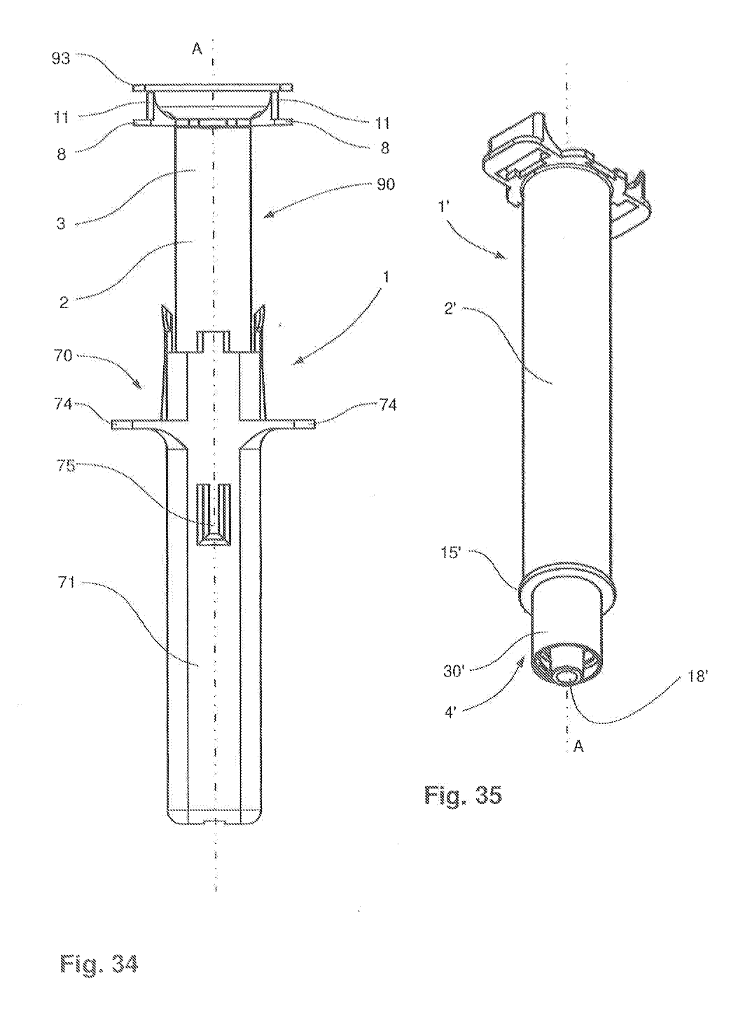

FIG. 34 shows an external view of the dispensing device from FIG. 24, wherein the protective device is in a protective position;

FIG. 35 shows an external view of a further embodiment of a main body of a dispensing device according to the invention with a protective device.

Features corresponding to each other are in principle provided with the same reference signs.

FIG. 1 snows a side view of a main body 2 of a dispensing device 1 according to the invention (see FIG. 23 for example). FIG. 2 shows a further side view of the main body 2 in a view perpendicular to the viewing direction of FIG. 1. FIG. 3 shows an external oblique view of the main body 2. FIG. 4 shows a detail of a dispensing area 4 of the main body 2. FIG. 5 shows a detail of a receiving chamber 5 for a closure element 30 (see FIGS. 6-10 for example) with a dispensing opening 18 of the main body 2. FIGS. 1 to 5 are described jointly below.

The main body 2 has a circular cylindrical, tubular portion 3 with a longitudinal axis A. The dispensing area 4 is formed at a front longitudinal end of the main body 2. In the text below, a direction along A 1 toward the dispensing area 4 is designated as “forward”, while an opposite direction is designated as “rearward”.

At a rear longitudinal end of the main body 2, wings 8 jutting out in a direction perpendicular to A are formed on opposite sides. In the direction of A, the wings 8 each have passages 9 in which latching edges 10 are formed for latching tongues 77 of a protective device 70. Protruding rearward in the direction of A, j support structures 11 are formed which, on the one hand, improve the stability of the wings 8 and, on the other hand, form abutments for a finger plate 33 of an actuating device 90 of the dispensing device 1 (see FIG. 23 for example). Moreover, the support structures 11 serve to protect the latching tongues 77 which, in a standby position of the protective device 70, protrude rearward through the passages 9 (in this connection see FIGS. 24-27 for example).

A receiving chamber 14 for receiving a fluid is formed in the cylindrical portion 3 (see FIGS. 16 and 19 for example). The receiving chamber 14 has a circular cylindrical cross section and is open at the rear longitudinal end of the main body 2. At the front longitudinal end, the receiving chamber 14 is closed by a lid-shaped closure piece 15, in front of which the dispensing area 4 is arranged. Starting from the closure piece 15, a supporting structure with cross-shaped ribs 16 extends forward, in the longitudinal direction A. Along the line of intersection of the ribs 16, a thickening 6 is formed in which a fluid channel 17 (see FIG. 19) extends in the longitudinal direction A to the receiving chamber 5 for the closure element 30. The thickening 6 is formed with a substantially circular cylindrical shape in the direction of A and is offset eccentrically with respect to the longitudinal axis A. The fluid channel 17 communicates with the receiving chamber 14.

The receiving chamber 5 is formed by an interior of a substantially circular cylindrical tube portion 19 which joins the ribs 16 in the forward, direction and is arranged coaxially with respect to the longitudinal axis A. Toward the front, the receiving chamber 5 is open substantially across its entire cross section. A bearing pin 7 for the closure element 30 is arranged coaxially with respect to A in the receiving chamber 5. On the bearing pin 7, a dispensing opening 18 of the main body 2 is arranged at which the fluid channel 17 opens to the outside. The dispensing opening 18 is formed on the front face of the bearing pin 7 eccentrically with respect to the longitudinal axis A. The front face of the bearing pin 7 has two ramp portions 20 ascending in the longitudinal direction A. The dispensing opening 18 is arranged on one of the ramps 20. The ramps 20 have the function of increasing a pressure between the corresponding portions of the closure element 30 and the front face of the bearing pin 7 when the closure element 30 inserted into the receiving chamber 5 is rotated. A better sealing action in the area of the dispensing opening 18 can be achieved in this way.

As a front end of the tube portion 19, two wings 22 protruding like flanges are formed on the outside and are arranged lying opposite each other with respect to the longitudinal direction A. The wings 22 form bayonet-like locking means, which can be brought into engagement with correspondingly configured looking means of a further device (in this connection see FIGS. 14 and 19 for example).

In the receiving chamber 5, latching notches 23 oriented in the longitudinal direction A are formed peripherally on an inner wall of the tube portion 19. The latching notches 23 are shaped and arranged in such a way that, when the closure element 30 is inserted, pawls 35 of the closure element 30 engage in the latching notches 23 and can thus block a relative rotation of main body 2 and closure element 30 in an undesired direction of rotation. The latching notches 23 and the pawls 35 thus form parts of a blocking device of the dispensing device 1.

In a radial direction with respect to A, the tube portion 19 has two passages 24 in which abutments 34 of the closure element 30 are arranged when tire latter is inserted into the receiving chamber 5. The passages 24 extend over a limited azimuthal angle range, as a result of which, the relative rotatability between main body 2 and closure element 30 is limited. The passages 24 and the abutments 31 thus form parts of a limiting device of the dispensing device 1.

FIG. 6 snows a side view of the closure element 30 of the dispensing device 1. FIG. 7 shows a further side view of the closure element 30, in a view perpendicular to the viewing direction of FIG. 6, and FIG. 3 shows an oblique view of the closure element 30. FIG. 9 shows a plan view of the closure element 30 along the longitudinal axis A, and FIG. 10 shows a plan view in the opposite viewing direction. FIGS. 6 to 10 are described jointly below.

The closure element 30 has a portion 31 which is of substantially circular cylindrical, shape and which is adjoined in the forward direction by a frustoconical portion 32. Here, “forward” and “rearward” and an arrangement of the axis A relate to the longitudinal axis A of the main body 2 when the closure element 30 is arranged on the main body 2 in the state ready for operation (in this connection see FIGS. 11, 14 and 13 for example).

In the state ready for operation, the closure element 30 is arranged with the circular cylindrical portion 31 in the receiving chamber 5 (in this connection see FIG. 11). The circular cylindrical portion 31 has a receiving chamber 33, limited by a side wall 36, for the bearing pin 7 of the main body 2. The two projections 34, which lie opposite each other with, respect to A and jut out perpendicularly with respect to A, are arranged externally on the side wall 36 in the circular cylindrical portion 31. In order to limit a relative range of rotation between main body 2 and closure element 30, the projections 34 engage in the passages 24 of the tube portion 19 when the closure element 30 is arranged in the receiving chamber 5 in the state ready for operation (in this connection see FIG. 11). Between the projections 34, the side wall 36 of the circular cylindrical portion 31 is interrupted, from the direction of the rear, such that the projections 34 for introduction into the passages 24 can be pivoted resiliently inward, i.e. toward the longitudinal axis A.

The pawls 35 are arranged on the circular cylindrical portion 31 in an area at the transition to the frustoconical portion 32. The pawls 35 are designed here as resilient tongues which, as viewed from the front in the direction of A, extend substantially tangentially in the clockwise direction from the side wall 36 of the portion 31.

The frustoconical portion 32 has a jacket surface 37 narrowing conically toward the front and is closed off toward the front at a front face 38. At its base where it adjoins the circular cylindrical portion 31, the portion 32 is recessed in a stepped shape relative to the latter. An inlet opening 43 of a filling channel 47 of the closure element 30 is formed on the jacket surface 37.

The inlet opening 19 is arranged on a sealing rib 39 which, as a convex bead, extends all the way round the circumference of the frustoconical portion 32 in relation to the longitudinal axis A. Further circumferential ribs, e.g. for locking or for additional sealing, can be provided before and after the sealing rib 39 in the longitudinal direction A. The sealing rib 39 ensures that sufficient leaktightness can be ensured in the area around the inlet opening 49 when the frustoconical portion 32 is fitted in a seat of, for example, a transfer device for a fluid. In front of the sealing rib 39, on opposite sides with respect to the longitudinal axis A, grooves 44 oriented in the longitudinal direction are formed on the jacket surface 37. A rear end of the grooves 44 is spaced apart from the sealing rib 39. An outlet opening 41 of the dispensing channel 40 is formed on the front face 38. The out let opening 41 is arranged eccentrically with respect to A.

In the receiving chamber 33, a seat 43 for the bearing pin 7 is formed as a depression in a bottom of the receiving chamber 33. The bearing pin 7 is arranged with its front face in the seat 43 when the closure element 33 is located in the state ready for operation in the receiving chamber 5 (in this connection see FIGS. 15 and 18 for example). An inlet opening 42 of the dispensing channel 40 and an outlet opening 48 of the filling channel 47 are formed at the bottom of the seat 43. The two openings 42 and 48 are arranged eccentrically with respect to A, at the same radial distance from A and at an azimuthel angle of 90 degrees to each other. In particular, the opening 42 is arranged flush with the opening 41 in relation to A. At the bottom of the seat 43, two ramp portions 16 are arranged which ascend in the longitudinal direction A and which interact sealingly with the ramp portions 20 of the bearing pin 7.

FIG. 11 snows a side view of the dispensing area 1 of the dispensing device 1, with the closure element 30 arranged in tire receiving chamber 5. The closure element 30 is arranged with the portion 31 in the receiving chamber 5. The projections 34 in the circular cylindrical portion 31 engage in the passages 24 of the tube portion. 19 in order to limit a range of relative rotation between main body 2 and closure element 30. The frustoconical portion 32 protrudes forward from the receiving chamber 5 in the direction of A and beyond the tube portion 19. The frustoconical portion 32 forms a coupling means of the dispensing device 1 to permit coupling to a further device that has a corresponding seat (in this connection see FIGS. 12-13 for example).

The closure element 30 is provided with a cannula 45. The cannula 45 is inserted through the outlet opening 41 directly into the dispensing channel 40. Preferably, injection-moulding is directly effected around cannula 45 during the production of the closure element 30. In this case, the dispensing channel 40 is formed by the cannula 45 in the closure element 30. The cannula 15 extends forward, parallel to the longitudinal direction A, from the front face 38 (in this connection see also FIGS. 16 and 19).

FIG. 12 shows a protective cap 30 for the dispensing device 1, with a transfer channel 31 for delivery of a fluid. FIG. 13 shows a detail of a seat 33 of the protective cap 50 for a coupling means of the dispensing device 1. FIGS. 12 and 13 are described jointly below.

The seat 53 permits the insertion of the dispensing device 1 into the protective cap 50. The seat 53 is formed on an insertion opening of an elongate sleeve 54 for receiving the cannula 45. The seat 53 is designed in such a way that the dispensing device 1 can be inserted with the frustoconical portion 32 of the closure element 30 into the protective cap 50 (see FIGS. 15 and 16 for example). The frustoconical portion 32 thus forms a coupling means of the dispensing device 1. The sleeve 54 is designed to receive the cannula 45 mounted on the closure element 30. On its outside, the sleeve 54 is provided with longitudinal ribs, which stiffen an outer wall. In the inserted state, the sleeve 54 is arranged substantially coaxially with respect to the longitudinal axis A. Here, “forward” and “rearward” and an arrangement of the axis A relate to the longitudinal axis A of the main body 2 when the dispensing device 1 is inserted in the intended manner into the protective cap 50.

The seat 53 has a conical shape complementing the frustoconical portion 32. A sealing notch 55 is arranged extending ail the way round an inner face of the seat 53 with respect to A, in such a way that, upon complete insertion of the frustoconical portion 32, the sealing rib 39 of the latter sits sealingly in the sealing notch 55. If further ribs are present for locking or additional sealing, the seat 53 can have corresponding notches. The seat 53 likewise has longitudinal ribs 56, in the direction of A, which can engage as anti-rotation means in the grooves 44 of the frustoconical portion 32 (in this connection see FIGS. 16 and 19 for example). The frustoconical portion 32 can thus be inserted info the seat 53 in the direction of A, wherein the sealing rib 39 latches in the sealing notch 55.

A circumferential, plate-shaped flange 57 is formed externally on the protective cap 50, in a longitudinal area of the seat 53. Two locking means 58 are arranged on the flange 57 and complement the locking means of the main body 2, which are formed as wings 22. The locking means 58 are designed here as undercuts with respect to A, in such a way that the wings 22 can be brought into engagement with the locking means 58 by rotation of the main body 2 about A (i.e. the undercuts are open laterally in the direction of rotation). Wings 22 and undercuts 58 thus form a bayonet-like locking means against removal of the frustoconical portion 32 from the seat 53.

A filling opening 32 of the transfer channel 51 is formed in the sealing notch 55. The filling opening 52 is arranged in such a way that the inlet opening 19 of the filling channel 47 of the closure element 30 sealingly connects to the filling opening 52 when the frustoconical portion 32 is arranged in the seat 53. In the radial, direction perpendicular to A, the transfer channel 51 in the protective cap 50 continues in a connector nozzle 59. The connector nozzle 59 serves for connecting the protective cap 50 to a source of fluid or a transfer device for transferring a fluid, e.g. from a glass vial. A transfer device of this kind, or a transfer system of this kind with several transfer devices, is described in PCT/EP2012/065953. For this purpose, the connector nozzle 59 has a receiving chamber 62 for a corresponding coupling element of the source of fluid or of the transfer device (see FIGS. 15 and 18 for example).

The connector nozzle 59 is arranged substantially in the plane of the flange 57 and extends in the radial direction perpendicular to A. In addition, holding means 61 can be formed on the flange 57 in the direction of the connector nozzle 59, with which holding means 61 the protective cap 50 can be fixed or supported or otherwise mounted on an attached transfer device, for example.

Between the locking means 58, ramps 60 are formed on the flange 57 and extend in the azimuthal direction about A. The ramps 60 are designed in the shape of an arc of a circle with respect to A and have a height that increases in the direction of A. The height increase of both ramps 60 takes place with the same sense of rotation with respect to A. The ramps 60 are arranged, in such a way that, by rotation of the main body 2 about A, the wings 22 can slide onto the ramps 60. In this way, from the relative rotation about A between protective cap 50 and dispensing device 1, a force can be generated in the direction of A. This force serves to lift the frustoconical portion 32 from the seat 53, such that the dispensing device 1 can be easily withdrawn from the protective cap 50. In particular, a latching of the sealing rib 33 in the sealing notch 55 can easily be released in this way on account of the rotation, which is required anyway, of the main body 2 for releasing the engagement of the wings 22 in the locking means 58 (in this connection see also FIGS. 16 and 19).

FIG. 14 snows a detail of the dispensing area 4 of the dispensing device 1 provided with the protective cap 50, with the closure element 30 in a filling position. FIG. 15 shows a partial view of a cross section of the dispensing area 4 from FIG. 14, in a section plane that comprises A. FIG. 16 shows a cross-sectional view of the view from FIG. 14, in a plane that comprises A and is arranged perpendicular to the section plane of FIG. 15. FIGS. 14 to 16 are described jointly below.

The wings 22 are in engagement with the locking means 57 of the protective cap 50 and lock the dispensing device 1 against removal from the protective cap 50 in the direction of A. The frustoconical portion 32 of the closure element 30 is arranged in the seat 53.

As can be seen from FIG. 15, the inlet opening 49 of the filling channel 47 adjoins the filling opening 52 of the transfer channel 51. At the same time, the outlet opening 48 of the filling channel 47 adjoins the dispensing opening 18 of the main body 2. A fluid connection therefore exists between the transfer channel 51 and the receiving chamber 14 of the main body 2 of the dispensing device 1. This position of the closure element. 30 is designated here as the filling position, since a fluid delivered via the connector nozzle 59 can be filled into the receiving chamber 14 or can be drawn up from the dispensing device 1. In the connector nozzle 59, the receiving chamber 62 for a coupling means, e.g. a source of fluid, can be seen in cross section.

It will also be seen from FIG. 15 that the circular cylindrical portion 31 is arranged in the receiving chamber 5 of the tube portion 19, and the bearing pin 7 protrudes into the seat 43 at the bottom of the receiving chamber 33 of the closure element 30.

In FIG. 16, the cannula 43 extending eccentrically and parallel to A can be seen in the interior of the sleeve 54. The cannula 45 extends in the dispensing channel 40 substantially through the entire closure element 30. As can be seen from the area at the hearing pin 7, a fluid connection between the fluid channel 17 of the main body 2 and the dispensing channel 40 of the closure element 30 is interrupted in the filling position. The filling position thus corresponds to a closed position.

FIG. 17 shows the same detail as FIG. 14, wherein the closure element 30 is in a dispensing position, shortly before complete removal from the protective cap 50. FIG. 18 shows a partial view of a cross section of the dispensing area 4 from FIG. 17, in a section plane that comprises A. FIG. 19 shows a cross-sectional view of the view from FIG. 17, in a plane that comprises A and is arranged perpendicular to the section plane of FIG. 18. FIGS. 17 to 19 are described jointly below.

In contrast to FIGS. 13 to 16, the main body 2 is rotated about the longitudinal axis A by about 90 degrees with respect to the protective cap 50. Since the closure element 30 is arranged in the seat 53 in a rotationally secure manner (through the engagement of the ribs 56 in the grooves 44), the closure element 30 is also rotated about A with respect to true main body 2.

On the one hand, on account of the relative rotation about A, the dispensing opening 18 of the main body 2 reaches the inlet opening 42 of the dispensing channel 40 of the closure element 30. In this position, a continuous fluid connection is thus established between the receiving chamber 14 and the cannula 45. This position of the closure element 30 is designated here as dispensing position.

On the other hand, on account of the relative rotation, the wings 22 have beer; released from the locking means 58 and in so doing nave slid onto the ramps 60. As a result of the wings 22 sliding onto the ramps 60, the closure element 30 is lifted from the seat 53 over the main body 2 (in the direction of the arrow). The grooves 44 and the ribs 56 are here designed in such a way that, during this lifting movement, the rotational securing remains between closure element 30 and seat 53.

The relative rotation between main body 2 and closure element 30 is limited on account of the projections 34 arranged in the passages 24. It will also be seen from FIG. 18 that, in the dispensing position, a fluid connection between the filling channel 47 and the fluid channel 17 or the receiving chamber 14 is interrupted.

FIG. 20 shows a side view of a protective body 71 of a protective device 70 (see FIG. 23 for example) of a dispensing device 1 according to the invention. FIG. 21 shows a further side view of the protective body 71 from FIG. 20, and FIG. 22 shows a detail of the trigger means, designed as latching tongues 77, of the protective body 71 from FIG. 20. FIGS. 20-22 are described jointly below.

The protective body 71 comprises a tubular sleeve 72 with a substantially square cross section rounded off at the edges. It will be appreciated that this design of the cross section represents only one example from a large number of possible alternative designs. In particular, the cross section can also be circular, for example, and preferably adapted to the circular cylindrical portion 3 of the main body 2. At a front end in the longitudinal direction A, the protective body 71 has an opening 73 (see FIG. 24 for example) through which the tube portion 19 of the main body 2 of the dispensing device 1 can pass when the protective body 71 is arranged in a standby position on the main body 2. The longitudinal axis A relates to the position of the longitudinal axis A of the main body 2 when the protective body 71 is arranged on the main body 2.

In a rear area, protruding wings 14 lying opposite each otter and perpendicular with respect to A are formed as a linger flange on the outer surface. The wings 74 are arranged in such a way that they are oriented corresponding to the wings 8 of the main body 2 when the protective body 71 is arranged on the main body 2.

Pairs of latching tongues 75 and 76 are arranged on the side walls of the sleeve 72. The two latching tongues of each of the pairs are arranged on side walls of the sleeve 72 lying opposite each other with respect to A, wherein a latching edge arranged at a free end of the latching tongues protrudes inward. The other pair is arranged on the other two opposite side walls. The pair 75 has its free end protruding forward, while the pair 76 is arranged farther forward in the longitudinal direction A on the protective body 71 and has its free ends protruding rearward. The free ends of the latching tongues 75 and 76 are arranged with respect to each other on the protective body 71 in such a way that, in a protective position of the protective body 71, i.e. when the latter is moved forward with respect to the main body 2 (in this connection see FIG. 34), the latching tongues 75 and 76 can latch on a protruding edge of the closure plate 15 on both sides with respect to the longitudinal direction A. The latching tongues 75 block the protective body 71 in the forward, direction, while the latching tongues 75 block it in the rearward direction. The protective body 71 is thus locked firmly on the main body 2 in the protective position. It is ensured in this way that the dispensing device cannot be used again after the protective device 70 has been transferred to the protective position.

A longitudinal area of the sleeve 72 in front of the latching tongues 76 is dimensioned in such a way that the cannula 45 is arranged completely in the interior of the sleeve 72 in the protective position.

In an end portion of the sleeve 72 behind the wings 74, a rearwardly protruding latching tongue 77 is formed on each of the side walls on which the wings 74 are arranged. The latching tongues 77 are of identical design, for which reason only one of the latching tongues 77 is described below by way of example.

The latching tongue 77 protrudes with its free longitudinal end beyond the sleeve 72 in the direction of A, and its free end can be pivoted with respect to the sleeve 72 toward the longitudinal axis A and away from the latter. The free longitudinal end of the latching tongue 77 has in each case two wings 78, which are separated from each other by a gap 79 that narrows toward the sleeve 72, i.e. in the forward, direction. On account of the gap 79, the wings 78 can be moved relative to each other in a direction perpendicular to A, in particular toward each other. The wings 78 can be connected resiliently to the rest of the latching tongue 77 such, that, after being deflected, they return to a starting position.

At an end directed toward the sleeve 72, the wings 78 each nave a shoulder 80, which serves as a latching edge of the latching tongue 77 for engaging on the latching edges 10 of the main body 2. The latching tongues 77 form a trigger means of the protective device 70.

In a projection in the direction of A, the wings 78 protrude outward with respect to A beyond the rest of the latching tongue 77. The wings 78 nave a dish shape, which is curved outward with respect to A. The wings 78 thus form a widening at the end of the latching tongues 77. This widening is deformable on account of the gap 94. On the other two side walls, wall portions 81 extend rearward beyond the sleeve 72 and, at least in the standby position, can engage in corresponding guide grooves on the main body 2.

FIG. 23 shows an external view of a dispensing device 1, which has a main, body 2 provided with the protective device 70. FIG. 24 shows a cross-sectional view of the view from FIG. 23, in a plane along which the wings 8 and 74, respectively, are oriented and in which the longitudinal axis A is arranged. FIG. 25 shows a partial view of the cross section from FIG. 24 at a rear end of the dispensing device 1. FIG. 26 snows a cross-sectional view in a section plane in the area of the latching tongue 77, as is indicated in FIG. 25. FIGS. 23 to 26 are described jointly below.

An actuating device 90 of the dispensing device 1 comprises a piston 91, a piston rod 92 connected, rigidly to the piston 91, and a dish-shaped finger plate 93. The dish-shaped finger plate 93 has an outer face 95 curved convexly toward, the main body 2. The piston 91 and the finger plate 93 are arranged at opposite longitudinal ends of the piston rod 92. The actuating device 90 is inserted completely into with the piston 91 into the receiving chamber 14 of the main body 2, wherein a longitudinal axis of the piston rod 92 is arranged coaxially with respect to the longitudinal axis A. The actuating device 90 is dimensioned in such a way that, in this position, the finger plate 93 bears on the support structures 11 of the main body 2.

The protective body 71 is pushed from the front over the main body 2, such that the tube portion 19 with the closure element 30 arranged therein, and the cannula 45 arranged thereon, protrudes forward in the longitudinal direction A through the opening 73. A helical spring 82, supported on the closure plate 15, is arranged between the main body 2 and the protective body 71. The tube portion 19 serves here as a seat for the helical spring 82.

The latching tongues 77 are guided through the passages 9 in the wings 8 of the main body 2 and are supported, i.e. locked, with their latching edges 80 on the latching edges 10 of the main body 2. The wings 78 protrude through passages 94 in the finger plate 93, which passages 94 extend in the direction of A through the finger plate 93. The passages 94 are dimensioned in such a way that, in this position, the wings 78 bear on an outer edge of the passages 94 with respect to A. The engagement, of the latching tongues 77 on the latching edges 10 of the main body 2 cannot be released on account of the wings 78 being supported on the edge of the passages 94. The finger plate 93, with the passages 94 formed thereon, thus forms a securing means of the dispensing device 1, with which securing means the protective device 70 can be brought to a secured state. A rearmost area of the wings 78 in the direction of A extends beyond a clear cross section, of the passages 94 in a projection along A.

The protective body 71 is thus situated in a standby position, wherein the protective device 70 is in a secured state.

FIG. 27 shows an external view of the dispensing device 1, wherein the protective device 70 is in an unsecured state. The dispensing device 1 is partially drawn up. FIG. 28 shows a partial view of a cross section at a rear end of the dispensing device 1. FIG. 29 shows a cross-sectional view in a section plane in the area of the latching tongue 77, as is indicated in FIG. 28. FIGS. 27 to 29 are described jointly below.

The finger plate 93 is moved together with the piston rod 92 and the piston 91 rearward from the main body 2 in the direction of A. The wings 78 of the latching tongues 77 are released from the passages 94 of the finger plate 93. The wings 78 of a latching tongue 77 have been forced toward each other so as to be able to slide through the corresponding passage 94. The gap 79 permits the necessary deformation of the widening of the latching tongue 77 formed by the wings 78. After being released, the wings 73 are returned again to the original position.

The latching tongues 77 are supported with the latching edges 80 on the latching edges 10 of the main body 2. Since the wings 78 are no longer supported on the passages 94, the latching tongues 77 can be moved or curved outward, i.e. in the radial direction away from the longitudinal axis A. With the securing means formed by the finger plate 93 with the passages 94 formed thereon, the protective device 70 can thus be brought to an unsecured, state.

The protective body 70 is thus still situated in the standby position, although the protective device 70 is in an unsecured state ready for triggering.

FIG. 30 shows an external view of the dispensing device 1 at the moment the protective device 70 is triggered. FIG. 31 shows a partial view of a cross section, at a rear end of the dispensing device 1. FIG. 32 shows a cross-sectional view in a section plane in the area of the latching tongue 77, as is indicated in FIG. 31. FIG. 33 shows an enlarged detail, of the depiction in FIG. 31, in the area of the wings 78 of one of the latching tongues 77. FIGS. 30 to 33 are described jointly below.

The closure element 30 is situated in a dispensing position, wherein the piston 91 is inserted substantially completely into the receiving chamber 14 of the main body 2. The dish-shaped linger plate 93 is moved toward the main body 2 along A, such that an outer face of the finger plate 93 abuts against the wings 73 of the latching tongues 77 from the rear.

Since the rearmost area of the wings 78 in the direction of A extends beyond a clear cross section of the passages 94 in a projection along A, the wings 78 slide onto the outer face 95 of the finger plate 93. If the finger plate 93 is pressed farther toward the main body 2, the convexly shaped outer face 95 of the finger plate 93 forces the wings 78 outward in the radial direction away from A. The outer face 95 thus forms a control face for triggering the latching tongues 77. The passages 9 in the wings 8 of the main body 2 are accordingly dimensioned for this purpose, such that the latching tongues 77 can be moved sufficiently far outward. In this way, an engagement of the latching edges 80 on the latching edges 10 is released and the protective device 70 is triggered.

After the release from the latching tongues 77, the protective body 71 is no longer locked on the main body 2 and is in principle free to be pushed, forward, with respect to the main body 2, to the protective position by the spring force of the spring 62. It will be appreciated, that a user can hold the protective body 71 at the finger flange 74 in the standby position even when the protective device 70 is already triggered.

FIG. 34 snows an external view of the dispensing device 1, wherein the protective device 70 is in the protective position.

The protective body 71 is shifted fully forward along A relative to the main body 2, such that the tubular portion 3 of the main body 2 lies substantially free. The protective body 71 is locked on the closure plate 15 via the pairs of latching tongues 75 and 76. The cannula 45 is arranged completely inside the protective body 71, such that there is no risk of injury to a user. The finger plate 93 is lowered completely onto the support structure 11, wherein the piston 91 is moved fully forward in the receiving chamber 14. The dispensing device 1 is thus emptied after one use. Since the protective body 71 is locked on the main body 2, the dispensing device 1 can no longer be used again and has to be discarded.

FIG. 35 snows a further embodiment of a main body 2 of a dispensing device 1′ shown incompletely (without actuating device and protective device), which main body 2′ is suitable for use with a protective device 70.

At least from the rear, and as far as a closure plate 15′, the main body 2′ is of identical design to the main body 2 of the dispensing device 1. Instead of the closure element 30, a conventional Luer look coupling 30′ is formed in the dispensing area 4′. A dispensing opening 18′, which is formed on a front face of a cone of the coupling 30′, communicates with a receiving chamber in the main body 2′ via an internal fluid channel. The main body 2′ can be provided with a protective device 70 in the same way as the dispensing device 1. In this case, the opening 73 of the protective body 71 is dimensioned in such a way that the luer coupling 30′ can protrude at least partially through it. The Luer coupling 30′ can serve as a seat for the helical spring 82. From the above description, it will be immediately apparent how the protective device 70 is to be arranged on the main body 2′.

1-23. (canceled)

24. A dispensing device for dispensing a fluid comprising:
a) a main body with a longitudinal axis,
b) a receiving chamber formed in the main body for the fluid,
c) a dispensing area, which is designed for the arrangement of a cannula communicating with the receiving chamber, or in which a cannula communicating with the receiving chamber is arranged, and through which the fluid can be dispensed from the receiving chamber,
d) wherein the dispensing device comprises a protective device with a rigid protective body arranged on the main body,
e) the protective body is designed in such a way and is movable with respect to the main body between a standby position and a protective position in such a way that the cannula arranged or able to be arranged in the dispensing area is completely exposed in the standby position, and the protective body, in the protective position, extends completely from the main body over the cannula arranged or able to be arranged in the dispensing area,
f) a trigger means is present which can be triggered directly or indirectly by a user and with which a transfer of the protective body from the standby position to the protective position can be triggered,
wherein a securing means is present with which the protective device can be brought to a secured state and an unsecured state, and the protective device, in the secured state, is secured against a triggering of the transfer from the standby position to the protective position, and, in the unsecured state, the transfer can be triggered.

25. The dispensing device as claimed in claim 24, wherein the securing means is designed in such a way that, in the secured state of the protective device, the trigger means is secured by the securing means against being triggered.

26. The dispensing device as claimed in claim 24, wherein the trigger means comprises at least one latching tongue which is formed on the protective body and, in the standby position of the protective body, is latched on a latching edge of the main body.

27. The dispensing device claimed in claim 24, wherein the dispensing device has an actuating device with a piston which is movable in the receiving chamber and which is movable from an inserted position to a retracted position in order to introduce the fluid into the receiving chamber, and is movable in the opposite direction in order to discharge the fluid, and the actuating device is formed as a securing means in such a way that the protective device can be brought to the secured state in the inserted state of the piston.

28. The dispensing device as claimed in claim 27, wherein the actuating device is formed in such a way that, upon the transfer of the piston from the retracted position to the inserted position, the trigger means can be triggered.

29. The dispensing device as claimed in claim 28, wherein the trigger means can be triggered in a constrained manner.

30. The dispensing device as claimed in claim 27, wherein the securing means is formed in such a way that, in the retracted position of the piston, the protective device is in the unsecured state.

31. The dispensing device as claimed in claim 30, wherein the protective device is in the unsecured state by a constraint.

32. The dispensing device as claimed in claim 27, wherein at least one of the securing means and the trigger means has a blocking means, which is designed in such a way that a restoration of the operative connection of the secured state is prevented once this operative connection is canceled.

33. The dispensing device as claimed in claim 27, wherein the actuating device has a finger plate for moving the piston, and the finger plate is connected to the piston and is designed as a securing means.

34. The dispensing device as claimed in claim 33, wherein the securing means is designed as at least one passage which is formed in the finger plate and through which the trigger means can protrude if the piston is in the inserted position, the passage (94) has an abutment that blocks the trigger means (77) in a trigger direction, and the abutment is arranged with respect to the latching tongue (77), and also to the latching edge (10) of the main body (2), in such a way that the latching tongue (77) can be fixed on the latching edge (10) by the abutment.

35. The dispensing device as claimed in claim 34, wherein the passage has an abutment that blocks the trigger means in a trigger direction, and the abutment is arranged with respect to the latching tongue (77), and also to the latching edge (10) of the main body (2), in such a way that the latching tongue (77) can be fixed on the latching edge (10) by the abutment.

36. The dispensing device as claimed in claim 35, wherein the abutment is arranged with respect to the latching tongue, and also to the latching edge of the main body, in such a way that the latching tongue can be fixed on the latching edge by the abutment.

37. The dispensing device as claimed in claim 27, wherein the actuating device has a control face which, during the transfer of the piston from the retracted position to the inserted position, cooperates with the trigger means in such a way that the trigger means is movable or is moved from the control face in a triggering direction.
